(19) United States
(12) Patent Application Publication
(10) Pub. No.: US 2025/0114509 A1
(43) Pub. Date: Apr. 10, 2025

(54) Arteriovenous Graft System

(71) Applicants: Technische Universiteit Delft, Delft (NL); Academisch Ziekenhuis Leiden, Leiden (NL)

(72) Inventors: Nicholas Andrew White, Delft (NL); Tim Horeman, Delft (NL); Joris Ivo Rotmans, Delft (NL); Koen Elzert Adriaan van der Bogt, Delft (NL)

(21) Appl. No.: 18/982,672

(22) Filed: Dec. 16, 2024

Related U.S. Application Data
(63) Continuation of application No. PCT/NL2023/050351, filed on Jun. 28, 2023.

(30) Foreign Application Priority Data
Jul. 26, 2022 (NL) 2032601

(51) Int. Cl. A61M 1/36; A61M 39/22

(52) U.S. Cl. CPC A61M 1/3655; A61M 39/22; A61M 2039/226; A61M 2205/3317

(57) ABSTRACT

An arteriovenous graft system including an arteriovenous graft with a valve device. The valve device has an open state in which fluid flow through the graft is possible, and a closed state in which the fluid flow through the arteriovenous graft is blocked. The graft system includes an actuator device to actuate the valve device between the closed state and the open state. A transmission cable connects the actuator device with the valve device. The actuator device includes at least one movable magnet that connects to a rotatable plate which is equipped with a spiralled groove. The spiralled groove receives a pin that is movable in a linear path. The pin is connected to the transmission cable so as to arrange that motion of the magnet translates into a linear displacement of the pin and the transmission cable connected thereto.

### CROSS-REFERENCE TO RELATED APPLICATIONS

This application is a continuation of International Patent Application No. PCT/NL2023/050351, filed Jun. 28, 2023, entitled “ARTERIOVENOUS GRAFT SYSTEM”, which claims priority to and the benefit of Netherlands Patent Application No. 2032601, entitled “ARTERIOVENOUS GRAFT SYSTEM”, filed Jul. 26, 2022, and the specifications and claims thereof are incorporated herein by reference.

### BACKGROUND OF THE INVENTION

### Field of the Invention

The invention relates to an arteriovenous graft system comprising an arteriovenous graft provided with a valve device, wherein the valve device has an open state, in which fluid flow through the graft is possible and a closed state, in which the fluid flow through the arteriovenous graft is blocked, wherein the graft system comprises an actuator device to actuate the valve device between the closed state and the open state, and wherein a transmission cable connects the actuator device with the valve device.

### Background

An arteriovenous graft system may be applied in hemodialysis patients. Hemodialysis is the process to filter a patient's blood and remove toxins with the aid of an extracorporeal dialysis machine. One end of the artificial graft is connected to an artery while the other end is connected to a vein, and the graft is typically placed either in the leg or arm of a patient. The artificial arteriovenous graft is used as a shunt to avoid complications with hemodialysis patients that require peripheral vascular access, as the complication rate is extremely high due to the constantly elevated and turbulent flow over the shunt.

WO2020/055247 discloses an arteriovenous graft system for subcutaneous placement, wherein the actuator device may be wirelessly controlled externally of the patient to move the valve device between the first position and the second position. This wireless control may for example use an electrical or magnetic field with which the actuator device may be controlled without direct contact. A magnetic field may for example be provided by a permanent magnet.

WO2020/055247 leaves open how the magnetic field operating on the actuator device may be realized in practice, and fails to disclose how to answer to specific requirements such as how to provide a stable position of the valve device in every position between the open state and the closed state.

Reference to prior publications in this application is provided as a matter of giving a more complete background and is not to be construed as an admission that such publications are prior art for purposes of determining the patentability of the present invention.

### BRIEF SUMMARY OF THE INVENTION

It is therefore one of the objects of the present invention to secure that the valve device can be stably positioned in every position between the open state and the closed state.

It is a further object of the invention to provide a lock to the valve device when it is in the closed position.

Still a further object of the invention is that the valve device will be non-backdrivable.

Yet a further object of the invention is that there will be a high transmission ratio between the actuator device and the valve device.

These and other objects of the invention are achieved with an arteriovenous graft system having features according to one or more of the appended claims.

According to a first aspect of the invention the actuator device comprises at least one movable magnet, which at least one magnet directly or indirectly connects to a rotatable plate which is equipped with a spiralled groove, wherein said spiralled groove receives a pin that is movable in a linear path, wherein said pin is connected to the transmission cable so as to arrange that motion of the at least one magnet translates into a linear displacement of the pin and of the transmission cable connected thereto. With the arteriovenous graft system of the invention the graft can be kept stable in any selected position because the graft is non-backdrivable. Thus, the flow through the graft can be regulated to any desired flow.

Apart from the above objects, over-actuation of the at least one magnet can be prevented by arranging that the at least one magnet is sensitive to excitation by an external magnetic field driving the at least one magnet into a desired motion.

To improve accuracy of motion and sensitivity of the at least one magnet to be placed in motion, it is preferable that the actuator device comprises a ring of magnets that are movable along a circular path.

Preferably the ring of magnets provides an asymmetric magnetic field. Such an arrangement causes that a linkage with an external apparatus providing said external magnetic field is only possible by a predefined positioning of the external apparatus.

In a suitable embodiment the ring of magnets are oriented with neighbouring magnets having alternating north and south poles, except for at least one neighbouring pair of magnets having the same orientation of north and south poles.

Desirably the pin which is connected to the transmission cable is mounted on a follower element that is movable in a linear groove provided in a supporting baseplate of the actuator device.

Reliable maneuvering of the magnets is promoted by arranging that the ring of magnets is kept in a predefined position with respect to each other by placement of the magnets in a magnet holder that is equipped with receptacles for the magnets that are regularly distributed along a circular path.

Although it is possible that the magnet or magnets directly connect to the rotatable plate which is equipped with the spiralled groove (by direct mounting or through magnetism), it is preferred to provide this connection indirectly by arranging that the magnet holder is rotationally fixed with respect to the rotatable plate.

Suitably the actuator device, the transmission cable, and the arteriovenous graft provided with the valve device are equipped to be placeable subcutaneously in a human or animal.

Preferably the actuator device is provided with stitching holes to enable stitching the actuator device to subcutaneous tissue so as to prevent migrating of the actuator device.

In still another aspect of the invention which can be applied independent from all the other features disclosed herein, or in combination with any one of such other features, the transmission cable is provided with a loop, wherein said loop is preferably stabilised to prevent its loosening. This loop can be tailored to a desired distance between the valve device and the actuator device. This allows any traction on subcutaneous tissue and vessels to be limited, without interfering with the operation of the arteriovenous graft system. Placing a knot in the loop makes it stable and prevents the loop from coming loose.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1A is an illustration showing an isometric view of an arteriovenous graft system according to an embodiment of the present invention;

FIG. 1B is an illustration showing a detail of FIG. 1A, to note the graft together with the valve device;

FIG. 2 is an illustration showing an exploded view of an actuator device forming part of the arteriovenous graft system according to an embodiment of the present invention;

FIG. 3 is an illustration showing a preferred magnet configuration of an actuator device forming part of the arteriovenous graft system according to an embodiment of the present invention; and

FIG. 4 is an illustration showing an arteriovenous graft system of FIG. 1, wherein the transmission cable is provided with a loop, and said loop is stabilised to prevent its loosening, according to an embodiment of the present invention.

### DETAILED DESCRIPTION OF THE INVENTION

Whenever in the figures the same reference numerals are applied, these numerals refer to the same parts.

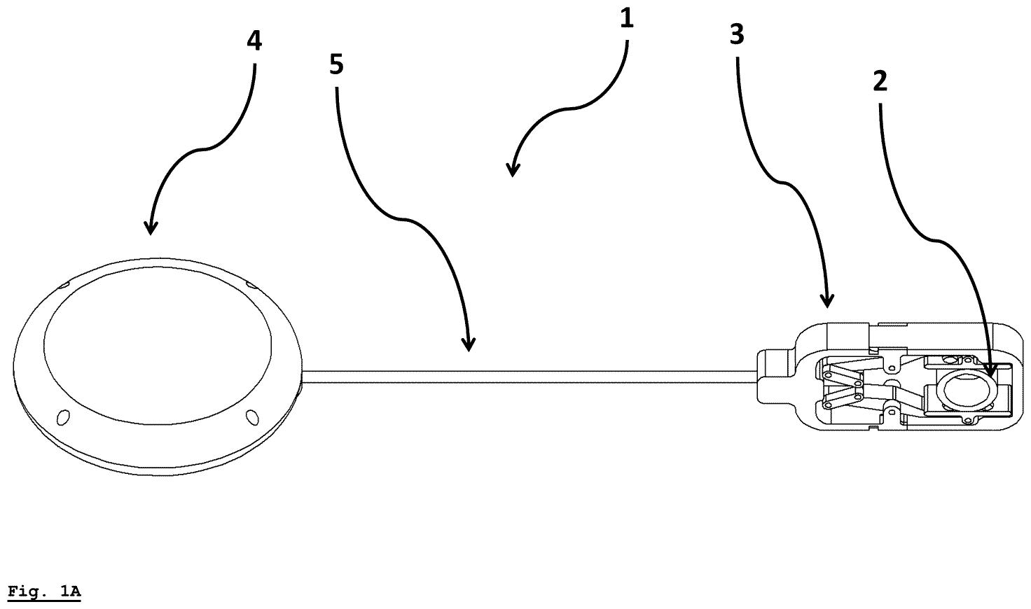

FIG. 1A depicts an arteriovenous graft system 1 comprising an arteriovenous graft 2 provided with valve device 3, wherein the valve device 3 is in an open state, in which fluid flow through graft 2 is possible. FIG. 1B shows in detail arteriovenous graft 2 and valve device 3 of FIG. 1A, wherein valve device 3 and the graft are in a closed state, in which the fluid flow through arteriovenous graft 2 is blocked.

The placement and operation of the combination of valve device 3 and arteriovenous graft 2 requires no further elucidation to explain the invention. The skilled person may refer to WO2020/055247 to understand this background of the invention.

Graft system 1 of the invention as explained hereinafter comprises actuator device 4 to actuate valve device 3 between the closed state and the open state, wherein transmission cable 5 connects actuator device 4 with valve device 3.

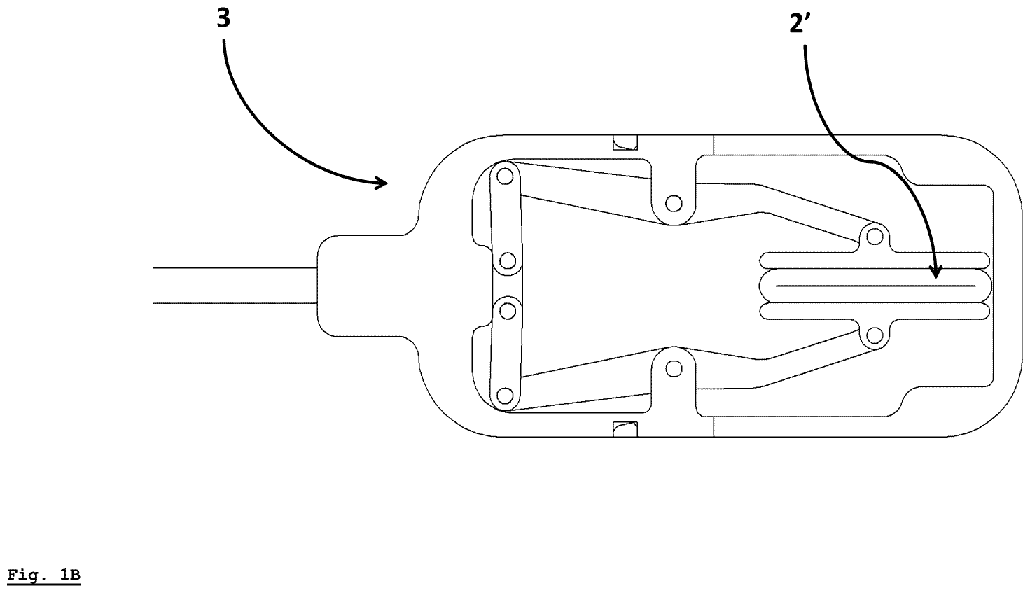

Turning now for an explanation of actuator device 4 to FIG. 2, it shows that actuator device 4 comprises at least one movable magnet 6. Actually, in the shown preferred embodiment actuator device 4 comprises ring of magnets 6 that are movable along a circular path. Ring of magnets 6 is preferably kept in a predefined position with respect to each other by placement of magnets 6 in magnet holder 9 that is equipped with regularly along circular path distributed receptacles 10 for magnets 6.

The at least one magnet or ring of magnets 6 connects to rotatable plate 7 which is equipped with spiralled groove 7′ (as shown at the underside of plate 7 visible in the right-hand part of this FIG. 2). The connection of the ring of magnets 6 to plate 7 is preferably provided by providing a rotationally fixed mounting of magnet holder 9 to plate 7.

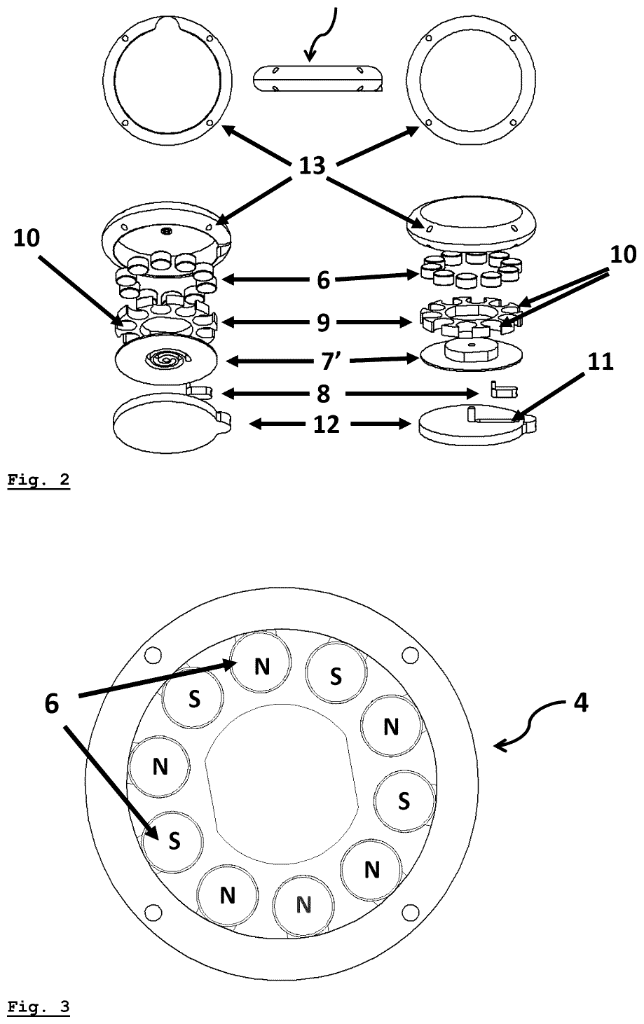

With reference to FIG. 3, it is shown that ring of magnets 6 provides an asymmetric magnetic field, by arranging that ring of magnets 6 are oriented with neighbouring magnets having alternating north and south poles, except for at least one neighbouring pair of magnets having the same orientation of north and south poles.

Turning back to FIG. 2, it is shown that spiralled groove 7′ at the underside of plate 7 receives pin 8 that is movable in a linear path. The movability of pin 8 along the linear path is preferably arranged by mounting pin 8 on follower element 8′ that is movable in linear groove 11 provided in supporting baseplate 12 of actuator device 4. Said pin 8 is connected to transmission cable 5 so as to arrange that motion of the at least one magnet or ring of magnets 6 translates into a linear displacement of pin 8 and likewise a linear displacement of transmission cable 5 connected thereto.

As already noted above, actuator device 4, transmission cable 5, and arteriovenous graft 2 provided with valve device 3 are equipped to be placeable subcutaneously in a human or animal. In order to operate arteriovenous graft system 1 of the invention, magnet or magnets 6 forming part of actuator device 4 is/are sensitive to excitation by an external magnetic field driving magnet or magnets 6 into a desired motion so as to actuate valve device 3 through intermediate transmission cable 5.

FIG. 2 shows that the actuator device is provided with stitching holes 13 to enable stitching actuator device 4 to subcutaneous tissue.

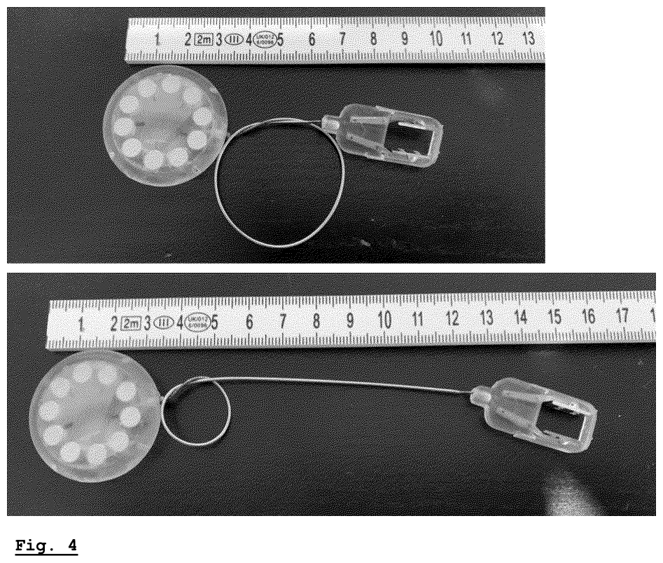

FIG. 4 shows that the transmission cable 5 is provided with a loop, wherein said loop is preferably stabilised to prevent its loosening. The loop can move freely to keep the distance between valve device 3 and actuator device 4 variable during everyday movements, thus limiting subcutaneous traction.

Embodiments of the present invention can include every combination of features that are disclosed herein independently from each other. Although the invention has been discussed in the foregoing with reference to an exemplary embodiment of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another. The terms, “a”, “an”, “the”, and “said” mean “one or more” unless context explicitly dictates otherwise.

1. An arteriovenous graft system comprising:
an arteriovenous graft comprises a valve device, wherein the valve device comprises an open state in which fluid flow through the graft is possible, and a closed state in which the fluid flow through the arteriovenous graft is blocked;
an actuator device to actuate the valve device between the closed state and the open state;
a transmission cable connecting the actuator device with the valve device;
wherein the actuator device comprises at least one movable magnet, which at least one movable magnet connects directly or indirectly to a rotatable plate comprising a spiralled groove, wherein said spiralled groove receives a pin that is movable in a linear path, wherein said pin is connected to the transmission cable so as to arrange that motion of the at least one magnet translates into a linear displacement of the pin and the transmission cable connected thereto.

2. The arteriovenous graft system of claim 1, wherein the actuator device comprises a ring of magnets that are movable along a circular path.

3. The arteriovenous graft system of claim 2, wherein the ring of magnets provides an asymmetric magnetic field.

4. The arteriovenous graft system of claim 2, wherein the ring of magnets are oriented with neighbouring magnets having alternating north and south poles, except for at least one neighbouring pair of magnets having the same orientation of north and south poles.

5. The arteriovenous graft system of claim 1, wherein the pin is mounted on a follower element that is movable in a linear groove provided in a supporting baseplate of the actuator device.

6. The arteriovenous graft system of claim 1, wherein the ring of magnets is kept in a predefined position with respect to each other by placement of the magnets in a magnet holder that is equipped with receptacles for the magnets that are regularly distributed along a circular path.

7. The arteriovenous graft system of claim 6, wherein the magnet holder is rotationally fixed with respect to the rotatable plate.

8. The arteriovenous graft system of claim 1, wherein the actuator device, the transmission cable, and the arteriovenous graft are placeable subcutaneously in a human or animal.

9. The arteriovenous graft system of claim 1, wherein the actuator device comprises stitching holes to enable stitching the actuator device to subcutaneous tissue.

10. The arteriovenous graft system of claim 1, wherein the magnet or magnets is/are sensitive to excitation by an external magnetic field driving the magnet or magnets into a desired motion.

11. The arteriovenous graft system of claim 1, wherein the transmission cable comprises a loop, wherein said loop is preferably stabilised to prevent its loosening.
